(19) United States
(12) Patent Application Publication
(10) Pub. No.: US 2009/0171195 A1
(43) Pub. Date: Jul. 2, 2009

(54) FUNCTIONAL IMAGING OF AUTOREGULATION

(76) Inventor: Randall L. Barbour, Glen Head, NY (US)

Correspondence Address: SCULLY SCOTT MURPHY & PRESSER, PC, 400 GARDEN CITY PLAZA, SUITE 300, GARDEN CITY, NY, 11530, US

(21) Appl. No.: 12/093,420

(22) PCT Filed: Nov. 13, 2006
(86) PCT No.: PCT/US06/44202

§ 371 Date: Aug. 19, 2008

Related U.S. Application Data
(60) Provisional application No. 60/735,800, filed on Nov. 11, 2005.

(51) Int. Cl. A61B 6/00; G06K 9/00

(52) U.S. Cl. 600/425; 382/131

(57) ABSTRACT

The present invention provides a method for detailed delineation of variation of autoregulation and more particularly tissue metabolism. These enhanced capabilities allow for new insights into factors impacting on body function, detection and monitoring of disease states, understanding of drug actions and other physiological effectors such as diet and physical exercise.

### BACKGROUND OF THE INVENTION

Autoregulation is the process whereby body tissues self regulate their local metabolic environments to maintain homeostasis. These processes involve a wide range of control mechanisms, including metabolic, hormonal and neural effectors. They also occur on different spatial scales, ranging from local cellular environments to control of whole body integrated mechanisms (e.g., regulation of blood pressure). This adaptive process, which often occurs on a fairly rapid time scale (sub-second to seconds), can also involve architectural adaptation, as is exemplified by the greater vascular density present in more metabolically active tissues.

There are many autoregulatory processes in the body that serve to maintain tissue metabolism in a state of balance and that serve as compensatory mechanisms when situations occur that produce imbalances in metabolite levels. Strenuous exercise, recovery from hypoxic states, response to hormonal and autonomic signals, and cardiovascular modulators (e.g., stretch sensors in the arotic arch, carotid bodies in the neck), among many others, all produce autoregulatory responses in one form or another. These responses are sensitive to specific metabolites (e.g., pH, Ca⁺⁺, CO₂, cyclicAMP, etc.) produced as a consequence of the induced metabolic states. Generally speaking these metabolites act on regulatory enzymes in a wide range of metabolic pathways that serve as control points in intermediary metabolism causing either positive or negative feedback signaling.

It is widely appreciated that derangements in autoregulatory processes lead to the onset of disease. For instance, failure in autonomic regulation leads to orthostatic intolerance, a condition wherein upon standing a subject incurs syncope. In addition, it is known that peripheral neuropathy can lead to poor control of blood pressure, L. Bernardi et al., “Reduction of 0.1 Hz microcirculatory fluctuations as evidence of sympathetic dysfunction in insulin-dependent diabetes,” Cardiovascular Research 34, 185-191 (1997). Similarly, autoregulatory imbalances in renal function are also known to produce a variety of metabolic disturbances, including electrolyte and water imbalances, and poor blood pressure control, among other pathologies.

Recognition of the importance of autoregulation has lead to the development of a variety of sensing technologies. These include methods for in vitro (e.g., laboratory analysis of blood samples) and in vivo analysis (e.g., imaging methods, ECG, pulse oximetry, noninvasive blood pressure measurements, etc.).

Among the imaging methods, an increasingly popular technique is the method of functional magnetic resonance imaging (fMRI). This method is sensitive to local changes in blood flow that accompany neuroactivation and can provide a time-series of images of a fraction of the hemoglobin species (i.e., deoxyhemoglobin), C. T. W. Moonen and P. A. Bandettini, Eds., Functional MRI (Springer-Verlag, Berlin, 2000).

Another neuroimaging technique sensitive to the influences of autoregulation is magnetoencephalography (MEG). This method also produces a time-series of images that are sensitive to the minute changes in magnetic fields produced in response to neuroactivity.

Still another imaging method gaining increasing acceptance is diffuse optical tomography (DOT). This method employs near infrared optical sources which, when combined with array sensing techniques and tomographic reconstruction methods, can also produce a time-series of images, R. L. Barbour et al., “Optical tomographic imaging of dynamic features of dense-scattering media,” J. Optical Society of America A 18, 3018-3036 (2001).

Despite the rapid improvements in sensing technologies, lagging has been the development of method and processes that is able to provide detailed description of autoregulatory processes.

Thus, it is an object of this invention to provide a method that is able to provide a detailed and accurate delineation of variation of Autoregulation and a process to enable said method.

It is another object of this invention to provide a method that is able to present said description via topographic, 2D tomographic image, 3D tomographic image, a time series or any combination thereof.

It is another object of this invention to provide a method comprises a step of comparing said image or time series for various application purposes.

The invention will be described in greater detail hereinafter by way of reference to the following drawings.

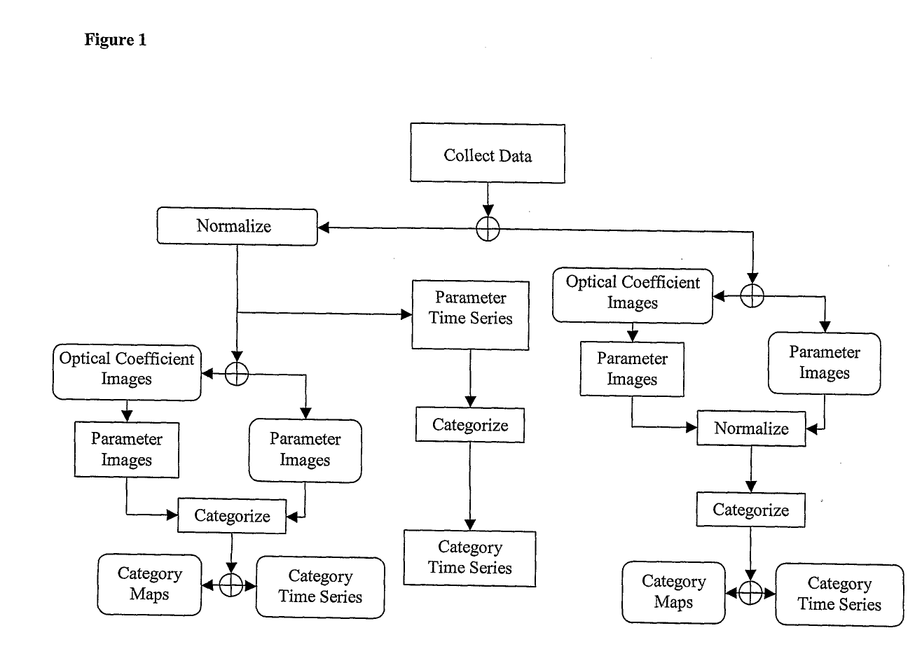

FIG. 1 illustrates a flow chart identifying the various data analysis approaches that can be implemented to characterize autoregulation in either an imaging or time-series domain.

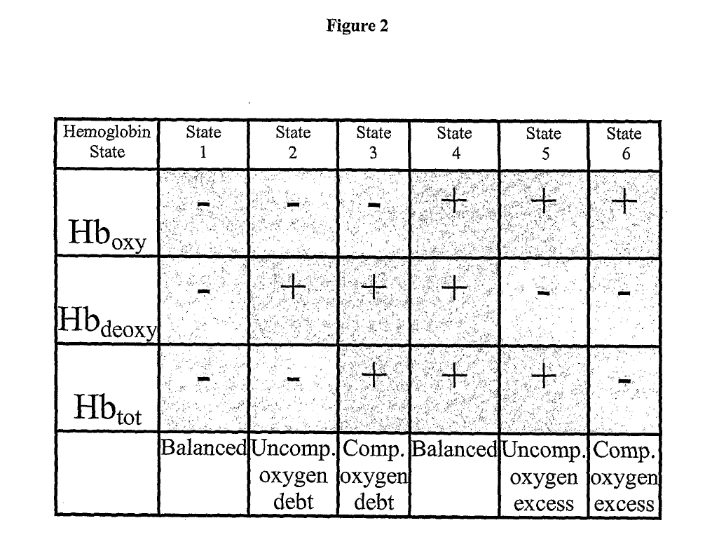

FIG. 2 represents a cycle of autoregulation of oxygen deliver to tissue through local variations in blood supply.

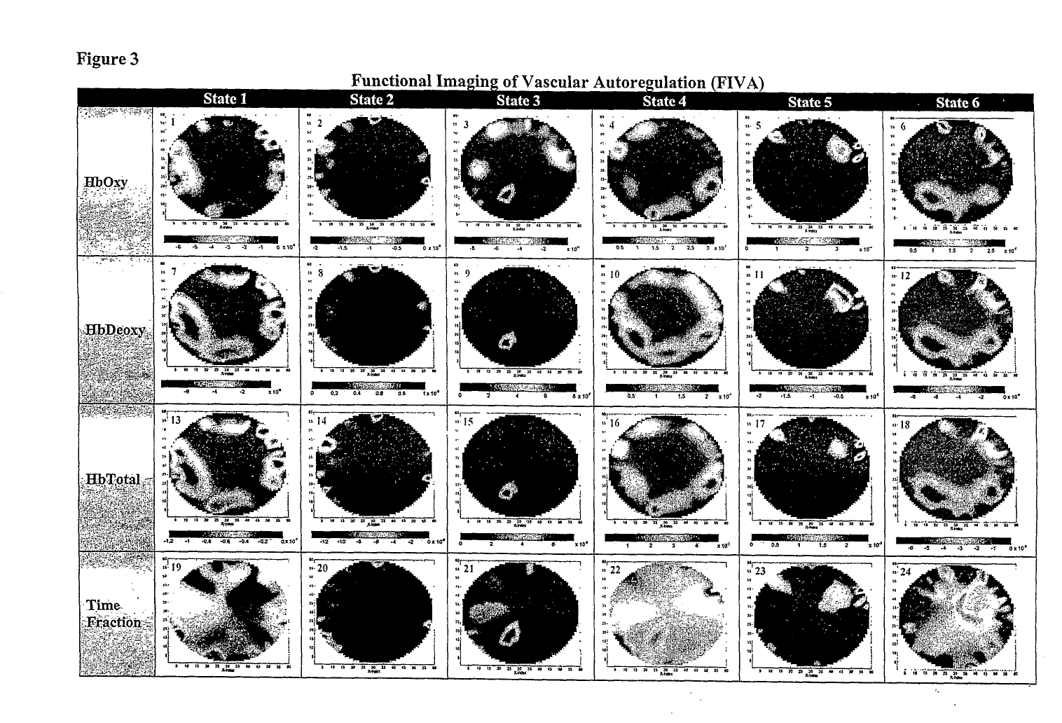

FIG. 3 are cross-sectional images of Functional Imaging of Autoregulation of a subject body during a cycle of hemoglobin regulation.

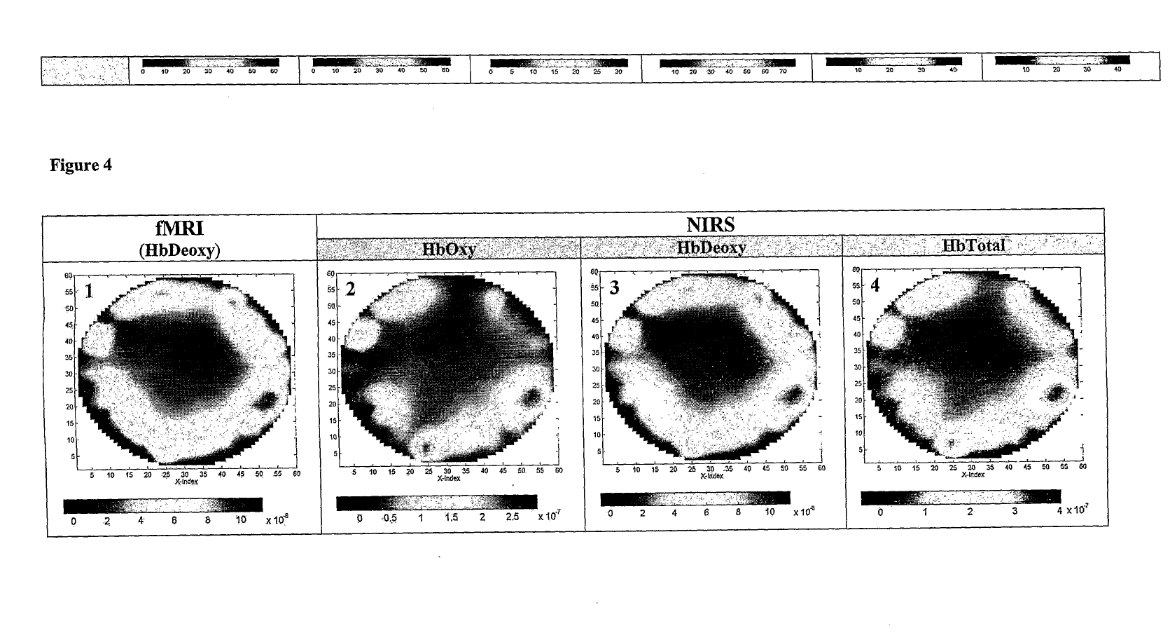

FIG. 4 is the cross-section image of fMRI and NIRS during a cycle of hemoglobin regulation.

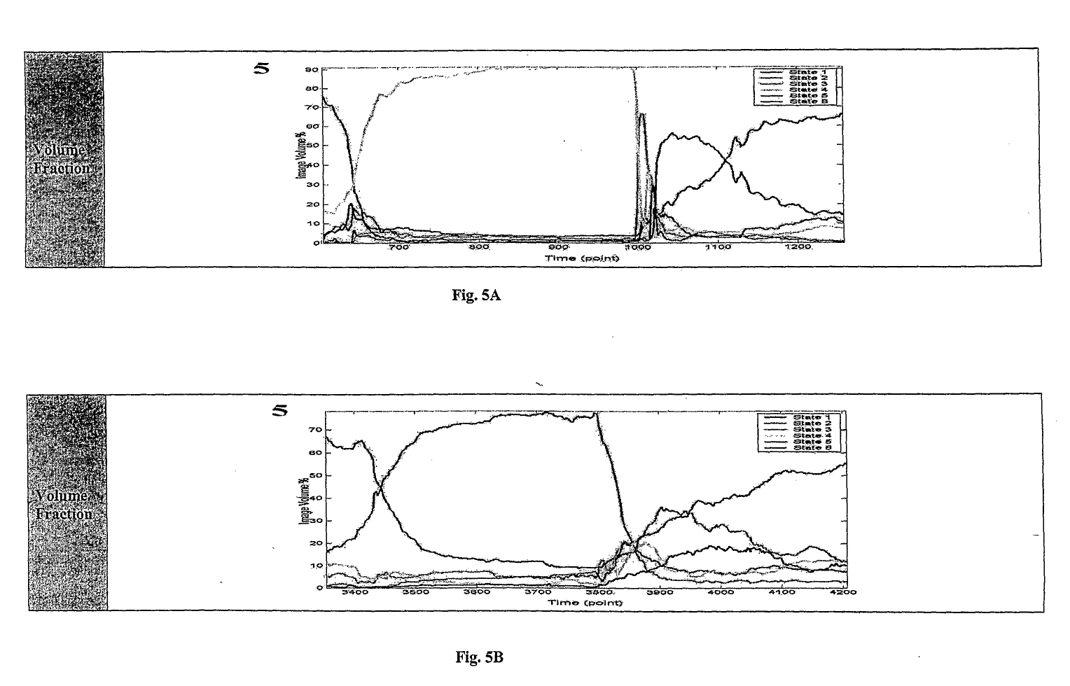

FIG. 5A is the time series of a vascular autoregulatory states in the experiment of 60 mm Hg. FIG. 5B is the time series of a vascular autoregulatory states in the experiment of 180 mm Hg.

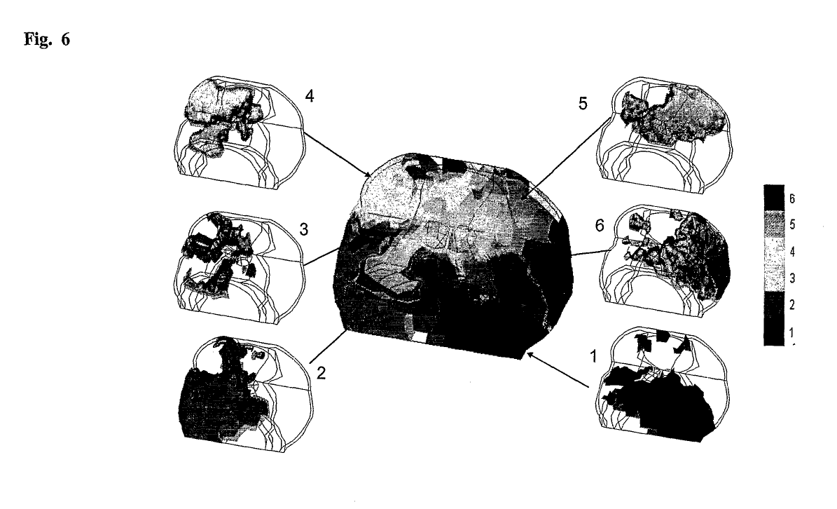

FIG. 6 is the volumetric image of a head of a rat showing six different states of the autoregulatory cycle at a single time point.

### DETAILED DESCRIPTION OF THE INVENTION

The monitoring of autoregulatory processes by noninvasive imaging methods embodies three independent elements. First, is the need for a sensing technology that provides for data collection speeds that are capable of capturing the relevant phenomenology. Second, is the need for a contrast agent, naturally occurring or otherwise, that actively participates in at least one step of the metabolic process comprising the autoregulation, or at least is sensitive to ensuing changes caused by autoregulation. For instance, in the case of vascular autoregulation, it would be useful to employ an oxygen sensing probe that also is responsive to the compensatory changes in blood volume that occur following oxygen debt. While, in principle, any of a number of sensing technologies could be employed, one of particular merit is near infrared optical methods. The key advantage here is that the contrast agent of interest is hemoglobin itself.

Still another important consideration that complements selection of the contrast agent as it relates to studies of autoregulation, is that accompanying its active participation in a particular process, this in itself can lead to separation of the total signal into experimentally definable fractions that are impacted by distinct physiological triggers. For example, in the case of hemoglobin, changes in metabolic demand can produces changes in its oxygenation level, which is usually followed by a hyperemic response.

The third element of monitoring autoregulatory processes is the need to process the data in ways that serve to delineate measurable quantities that enable the examination of physiological states that otherwise might be not be observable. For instance, in the case of vascular autoregulation, wherein it can be expected that variations in metabolic demand and supply can produce states having opposite influences on blood volume and its oxygenation level, measures that simply consider individual components (e.g., oxyhemoglobin) independent of others (i.e., deoxyhemoglobin, total hemoglobin) can lead to the loss of information owing in part to the limited spatial resolution offered by NIR imaging methods. This follows because the spatial blurring inherent to the method will produce cancellation of signals having opposite amplitudes. In contrast, the added delineation that follows by additional processing of the data into categories that are both experimentally observable and are closely tied to known physiological triggers assures that the indicated loss of information will not occur.

This invention is directed to a method comprising the steps of: applying at least one sensor to a portion of a subject's body; directing at least one energy source at a portion of the subject's body; detecting the emitted energy signal from at least one detector; processing said data; and producing at least one image or at least a time series or a combination thereof, to delineate variations of tissue metabolism, wherein the image can be a topographic, 2D tomographic, 3D tomographic map or any of the combination thereof.

In the application, image means a topographic, 2D tomographic or 3D tomographic map that reveals the spatial dependence of some parameter related to the considered autoregulatory state. Said image can either be a single image, a set of images of multiple parameters.

Derivative information means any information that can be derived from the images. One example can be a time-series of images that identify the time-dependence of the considered parameters.

Time series means a spatially integrated time dependence of a parameter related to the autoregulatory state, which can be shown in any kind of format.

Delineate means detailed sub-stage information for any cycle of tissue metabolism.

In one aspect of the invention, the energy source comprises at least one wavelength.

Energy sources can be any kinds of energy sources that are available for any kinds of devices. For example, the energy source can monochromatic (e.g., laser sources) or polychromatic (tungsten lamps, superluminescent diodes), light sources, and corresponding acoustic sources as appropriate among others, bio-related energy provider, or any combination thereof. And the energy source can be at any portion of a subject's body (e.g., inside, outside).

This invention also directs to a process for deriving information on tissue metabolism in a subject's body to produce at least one image or a time series comprising the steps of: 1) collecting data from at least one detector; 2) normalizing collected data to an experimental or computed mean value; 3) producing at least one parameter map having at least one pixel value from the normalized data using indirect imaging methods, or computing at least one image map from the normalized data and converting to at least one parameter map having at least one pixel value; 4) comparing pixel values of the parameter map in step (3) to their respective mean value and categorizing such values according to whether the parameter value is above or below its mean value; and 5) computing a parameter map of categorized pixel data and optionally producing a time series comprising a step of computing a spatial mean value for each parameter at each time point; wherein the image can be a topographic, 2D tomographic, 3D tomographic map or any combination thereof.

This invention also directs to a process for deriving information on tissue metabolism in a subject's body to produce at least one image, at least a time series, a combination thereof comprising the steps of: (1) collecting data from at least one detector; (2) producing at least one parameter map having at least one pixel value from the collected data using indirect imaging methods, or generating at least one image map from the collected data using said indirect imaging methods and converting to at least one parameter map having at least one pixel value; (3) normalizing pixel values in Step (2) to an experimental or computed mean value either after generation of the parameter map(s) or prior to conversion to said parameter map; (4) comparing pixel values of at least one parameter map in Step (3) to their respective mean value and categorizing such values according to whether the parameter value is above or below its mean value; and (5) computing a parameter map of categorized pixel value and optionally producing a time series comprising a step of computing a spatial mean value for each parameter at each time point, wherein the image can be a topographic, 2D tomographic, 3D tomographic map, or any combination thereof.

This invention also directs to a process for deriving information on tissue metabolism in a subject's body to produce at least one time series comprising the steps of: (1) collecting data from at least one detector, (2) normalizing collected data to an experimental or computed mean value followed by computation of a parameter value or computing the parameter value from collected data followed by normalization of the resultant time series; and (3) Comparing the values in Step (2) to their respective mean value and categorizing such values according to whether the parameter value is above or below its mean value.

A flow chart illustrating the processing scheme that serves to delineate various elements of tissue metabolism associated with autoregulatory states is shown in FIG. 1.

Following data collection data can be normalized, as indicated above, or not dependent on which branch of the flow chart is followed. Proceeding along the left branch, following normalization of the measurement data, images can be computed using any of a variety of indirect imaging techniques. Here we refer to class of methods generally known as algebraic reconstruction methods. These techniques often employ imaging operators based on physical models of radiation transport (e.g., diffusion equation, in the case of NIR imaging). It is well understood by those skilled in the art that these methods can be implemented to compute either a first-order reconstruction, or iterative recursive solutions can be sought. In the latter case, and for instances involving NIR imaging methods, it can be desirable to implement these so as to allow for computation of absolute optical coefficients (i.e., absorption and scattering coefficients). In this case, the above mentioned normalization should be performed following image reconstruction. In the case of first order reconstruction methods, normalization of the collected data can be performed either before or after image recovery. It is also understood that these methods can be implemented so as to produce images of optical coefficient maps (i.e., wavelength dependent absorption, scattering coefficients) from which parameter images (e.g., hemoglobin states) can be subsequently computed or alternatively the parameter maps can be directly computed (e.g., imaging with spectral constraints).

Following generation of the parameter images, the pixel data is then categorized according to the different states identified by the particular autoregulatory process under study. In the case of vascular autoregulation, this consists of six (6) categories, each comprising three elements (oxyHb, deoxyhb, totalHb). Having defined these categories, images of these can be directly produced by simply identifying their pixel time dependence. The derived image time series can be additionally processed, if desired to produce a time averaged image result such as is depicted in FIG. 3. Alternatively, by spatially integrating these images at each time point, a corresponding category time series can be produced such as is illustrated in FIGS. 5A and 5B.

The sequence of operations depicted in the right branch of FIG. 1 is different from the left only in the positioning of the normalization step. In many cases, the final result will not depend strongly on whether normalization is carried out before or after image formation. However, there are contexts where it would be preferable to apply the normalization after image formation rather than before. An example of this, i.e., when recovery of absolute optical coefficient values is attempted, is mentioned above. Another reason for possibly preferring to normalize after reconstruction is that this strategy gives more importance to detector channels with higher-amplitude data. In many cases there is a direct correspondence between the magnitudes of measured signals and the confidence that can be placed in them. Once the images are computed and normalized, the same processes of categorizing the parameter images, and of temporal and spatial integration, as described above are applicable here as well.

A third, not image-based strategy for processing collected data is depicted in the middle branch of FIG. 1. In this approach, the data from all detector channels are averaged together (for each measurement wavelength separately) to produce a small number of spatial mean time series. Using a theoretical formulation such as the modified Beer-Lambert law, the mean time series can be processed to yield spatially integrated hemoglobin-state time series. These time series can be processed, using the same categorization method as applied to images, to reveal spatially integrated time courses for the autoregulatory parameters of interest.

In this application, one aim of the analysis scheme is to express changes in autoregulatory state responses, it is convenient to consider these relative to some experimentally derived value or one that is computed based on, for example, a model prediction (e.g., state space modeling).

The present invention covers any possible process to achieve said goal. One embodiment contemplated by the present invention to achieve the result is to normalize the collected data, for each source-detector channel, to its corresponding temporal mean value. Normalization, serves as a simple classification scheme while also effectively reducing the dimensionality of the original data as absolute amplitude variations are removed. It is understood, however, that there are many other coefficient values that could be substituted for the normalization coefficient so as to emphasize changes with respect to some other parameter of interest (e.g., blood pressure, heart rate, etc.). The consideration here is similar in concept to employing linear regression methods to emphasize one response over another, or to remove (compensate) a particular feature.

In another embodiment of the invention, a sensor can be placed directly on the subject body or can be placed remotely from the subject's body or inside the subject's body or combined. By combine is meant that sensor can be used be place inside of the subject's body. The sensor measures a photo-acoustic signal, or modulation of light produced by focused ultrasound, or a fluorescent signal, or any combination thereof.

Preferably, the detection of tissue metabolism further comprises a contrast agent.

Preferably, the tissue metabolism is associated with hemoglobin.

In another aspect of the invention, an image is formed by a temporal, non-temporal mean or any combination thereof.

In another aspect of the invention, the collected data is a measure of a fraction of the incident energy or is a measure of incident energy that has been converted to another energy form or combined.

In another aspect of the invention, sensor can be any kinds of sensor that is available for any kinds of devices. For example, sensor measurements include silicon photodiodes, avalanche photodiodes, photo-multiplier tubes, charge coupled devices (CCD), charge inductive devices (CID), streak cameras, and corresponding acoustic sensing devices, any combination thereof.

In another aspect of the invention, any kinds of energy sources and sensors can be implemented various different ways. For instance, it well understood that optical and acoustic measurements can be made under continuous wave conditions (CW), wherein the source intensity is time invariant, or if modulated, the frequency of modulation is low compared to RF frequencies.

In one example, measurements can be made using frequency domain techniques wherein the amplitude of the source is varied in the RF range (e.g., 50-2000 MHz) and suitable adjustments are made to the detection electronics to permit sensing of the emitted signal (e.g., homodyne or heterodyne detection strategies).

A well known measurement technique is the use of ultra fast detection methods wherein the source is a ultrafast pulsed source (e.g., laser), and corresponding ultrafast detection methods are employed (e.g., streak camera).

Another technique useful in connection with the present invention is the detection of bioluminescent signals, wherein the energy source is produced inside tissue as a consequence of metabolic activity, thus functioning in a manner analogous to the contrast probe outlined above. It is understood that any combination of energy source sensor methods can be implemented.

In another aspect of the invention, any kinds of data collection schemes for devices can be used in this application. For example, probes can be employed to make direct contact with body tissues or measurements can be made remotely (i.e., non-contact). Another example can be acoustically combined methods wherein some appropriate conducting medium would be required (e.g., water). Still another example is in the case of data collection methods involving detection of fluorescence; use of an appropriate blocking filter would be required to isolate the fluorescent signal from the excitatory light.

In another aspect of this invention, any kinds of contrast agents can be employed allowing for investigation of autoregulatory processes so as to delineate various elements of tissue metabolism. It can be natural or synthetic.

For example, hemoglobin itself can be a contrast agent. This probe is particularly well suited for some applications. It is the principal species in the body responsible for oxygen transport to tissue; it undergoes distinct physiochemical state changes associated with oxygen binding that produce measurable changes in its optical properties; and it is normally confined to the vascular space thus enabling specific detection of changes in blood volume.

Equivalent measurements could be accomplished using a synthetic analogue of hemoglobin. Similar measurement could also be accomplished using optical probes that undergo spectral changes upon ligand binding. These comprise a large class of compounds that can undergo either absorption or fluorescent changes (including fluorescent lifetime). In addition, these compounds can be coupled to various targeting vehicles such as nanoparticles, macromolecules (e.g., monoclonal antibodies), liposomes, etc.

The nature of the ligand binding also comprises a large class of compounds. In many instances, these are low molecular weight compounds such as protons (pH), Ca⁺⁺, cyclic AMP, or intra- or extracellular ions. They can also comprise larger molecular weight species, such as components of intermediary metabolism (e.g., carbohydrates, amino acids, lipids, nucleic acids, hormones) or even macromolecules (e.g., membrane bound proteins, enzymes, RNA, DNA, plasma proteins, antibodies, etc).

Thus, by selecting the appropriate contrast agent (naturally occurring or synthetic), corresponding measurement approach (absorption, fluorescence, bioluminescence), energy source, detector/data collection scheme, together with appropriate data analysis strategies outlined below, a vast array of metabolic processes can be delineated that are linked to measurement of autoregulatory states.

In this application, any of a number of data collection schemes, energy sources and contrast agents can be implemented that meet the above criteria. For instance, whereas in the preferred embodiment for the application involving investigation of vascular autoregulation, the considered method of choice is near infrared diffuse optical tomography, it is expressly understood that either a photoacoustic, acousto-optical (i.e., acoustic modulation of light using focused ultrasound), or fluorescent measurement scheme could also be employed. In the case of the former, an acoustic sensor would be required instead of an optical sensor that would be required for either of the other mentioned optical methods. In cases where the considered data collection scheme leads to the formation of an image, it can be expected that more than one sensor and illumination site will be required. These measurements can involve using an array that generates multiple illumination-detection pairs or can involve a single source and detector that is repositioned about the target tissue in a manner executing a raster scan.

This invention can be applied in various kinds of autoregulation and tissue metabolism. One of the application is to delineate the information outlined in FIG. 2. For instance, in subjects who are candidates for vascular surgery, to install a fistula in support of kidney dialysis, it is not clear which areas on the forearm can reasonably support this procedure. Areas that are more hypoxic or have poor perfusion can be expected not to tolerate well the considered procedure. Also, in the case of breast cancer, knowing the state of tissue hypoxia, and its capacity to be oxygenated (e.g., by breathing 100% O₂), can influence the decision of whether radiation therapy is indicated. In the case of a stroke, the considered delineation of information afforded by the method described here, can be used to distinguish, for instance, between those regions of tissue continuing to experience oxygen debt (see States 2 and 3, FIG. 3) and hence may be subject to additional damage, from those that are can be potentially salvage (see, e.g., State 5 (reactive hyperemia), FIG. 3). By contrast, all of the indicated states would not be distinguishable by, for example, fMRI as they include both increases in the level of either deoxyhemoglobin or total hemoglobin.

Still other applications can involve measures obtained on exposed organs, naturally present or implanted, during surgery that serve as guides as to whether adequate perfusion is present.

The information content available from spatial maps of the sort shown in FIG. 1 can be significantly enhanced by obtaining these under conditions of specific provocation such as can be induced by maneuver or by a drug. These maneuvers can be targeted to manipulate either the vascular response (e.g., vasodilators, constrictors) or particular aspects of tissue metabolism (e.g., calcium inhibitors).

Psychiatric conditions, learning disorders in children, assessment of physical conditioning, detection of tumors, early diagnosis of diabetes, and many other disorders are all capable of imposing spatio-temporal distortions in the normal response of tissue to variations in the autoregulatory cycle. Thus assessment of this information can be used for diagnosis, prognosis, treatment monitoring and evaluation of the intended and unintended impact of drugs.

While many of the above applications measures associated with vascular autoregulation, through appropriate use of other contrast agents (naturally occurring or synthetic) many other elements of metabolism can be explored that may assist in disease diagnosis, prognosis or monitoring of treatment. For instance, one chromophore of interest is tissue water content. Yet another is glucose, which also has a measurable optical signal. Still others include myoglobin, lipid, bilirubin etc. Injectable contrast agents include use of indocynanin green, as well as the growing classes of fluorescent compounds that have NIR observable signals. The latter can be explored in a variety of ways. For instance, one class includes performing fluorescent resonance energy transfer (FRET) measurements. For these, the considered fluorescent probes can be made sensitive to any of a variety of chemical environments that occur in tissue. Other classes of probes than can be employed include those that are activateable in response to enzymatic activity or gene expression. In addition to use of exogeneous contrast agents, the considered scheme is also easily extended to explore the intended and unintended actions of pharmaceutical agents. This can prove extremely valuable in assisting in drug discovery. Still other practical applications involve manipulations to the body that serve to impact on the tissue-vascular response. These can comprise a wide class ranging from specific exercise regiments, diets, stresses, drugs, etc. In this manner, by monitoring these responses as delineated using the described method as outlined within, can serve to as a guide to optimize the desired outcome (e.g., weight loss, maximal physical performance, early detection of disease, optimal choice of drugs, etc.).

### EXPERIMENTAL RESULTS

To exemplify the process of Functional Imaging of Autoregulation, we present finding from a study wherein the considered autoregulatory phenomenon is the vascular response to mild hypoxia or cuff induced ischemia. The phenomenology of vascular autoregulation is well understood and involves active feedback mechanisms that adjust blood delivery to tissue, and hence oxygen delivery, so as to meet prevailing metabolic demand. In cases where, demand exceeds supply, the tissue will experience a brief period of oxygen debt, causing the release of tissue factors that serve to dilate the local microvasculature. This leads to enhanced perfusion. Should the oxygen debt be of sufficient magnitude, the resulting dilation of the vasculature can be more pronounced resulting in a hyperemia wherein oxygen supply exceeds demand. The resulting state of relative hyperperfusion leads to the washout of tissue factors that produce vasodilation leading to a return to normal perfusion levels with the restoration of oxygen supply to a level in balance with tissue demand.

To facilitate understanding of this phenomenology, we have listed the various stages of vascular autoregulation in FIG. 2 taking into account the different classes of experimentally definable states available when near infrared sensing methods are used. As outlined in Detailed Description, it is instructive to express the variations in autoregulatory states with regards to some mean value, for which here we have chosen the temporal mean associated with each of the measurable hemoglobin states. This produces quantities that are either above or below their respective mean. This classification, combined with an understanding of the associated functional changes that occur during vascular autoregulation suggests the pairing of states as outlined in FIG. 2. For presentation purposes we have color coded the sign of the hemoglobin states to distinguish those that are above or below its temporal mean value. Using the above described classification scheme, there are six unique pairing of hemoglobin states (oxyHb, deoxyHb, totalhb) that can be identified and are identified as States 1-6. In addition, these have been ordered in a manner consistent with the known phenomenology of vascular autoregulation, and under normal circumstances, can be viewed as a cyclical process wherein following State 6, the tissue returns to State 1.

To exemplify the information obtainable by the process of Functional Imaging of Autoregulation, the results shown in Experiment 1 below were acquired using the method of diffuse optical tomography and further processed as identified in the Detailed Description. In contrast to the richness of information identified in FIG. 3, applying the same analysis strategy as outlined above to data collected using the fMRI technique would limit exploration of vascular autoregulation to variations in only the deoxyhemoglobin. Similarly, applying of the method of diffuse optical tomography as it is usually practiced, without the additional analysis methods outlined in Detailed Description, is equivalent of integrating the values in FIG. 2 across each row. As will be shown, this integration can obscure valuable information.

### Experiment 1

Method: A human forearm is used as the subject body to study the tissue metabolism of hemoglobin in response to a 60 mm Hg pressure cuff inflation (mild hypoxia) for two (2) minutes and in response to a similar maneuver, but at 180 mm Hg pressure to produce ischemia. The method of Functional Imaging of Autoregulation (FIA) is used to describe the detailed variation of different states of hemoglobin during the cycle of autoregulation.

### Experiment 2

The head of a rat is used as the subject body to study tissue metabolism of hemoglobin. In this experiment a tether of optical fibers were attached to a head stage allowing the animal to move freely. The method of Functional Imaging of Autoregulation (FIA) is used to describe the detailed variation of different states of hemoglobin for a single time point.

Instrument: Data was acquired at 2.4 Hz for a period of 5-6 minutes using a DYNOT 232 imager (NIRx Medical Technologies) and includes the both provocation and recovery time. The time-series tomographic data sets were acquired using two (2) illuminating wavelengths (760, 830 nm).

Results:

FIG. 3 shows the results for each of the eighteen (18) hemoglobin fractions corresponding to the six (6) different autoregulatory states shown in FIG. 1, (panels 1-18), were computed for the mild hypoxia experiment. These were determined by first computing the image time-series associated with each of these sub states, followed by computing their temporal mean value over the 5-6 minute data collection period. Also shown in FIG. 3 (panels 19-24) is a spatial map identifying the pixel dependence of the fraction of the total time of observation (5-6 minutes) that was spent in each of the six (6) autoregulatory substrates. Note that the orientation of the 2D cross-sectional images shown is as follows: Top (dorsum), Bottom (Ventrum), Left (Medial), Right (Lateral). This view is generated when the cross-section of the arm is viewed in a caudal-rostral orientation.

A gross comparison of the findings in panels 1-18 reveals numerous qualitative and quantitative differences in tissue dependent features (note, that the color scale identifies Molar changes in Hb concentration). For instance, seen clearly in States 1 and 4 is the engorgement of near surface veins in response to application of 60 mm Hg pressure. This level of pressure is sufficient to induce venous occlusion, but not arterial occlusion, and hence can be expected to produce the venous engorgement seen. Supporting this finding is the observation that quantitatively, the magnitude of the response seen for State 4 (i.e., increase in blood volume but no change in its oxygenation level) is much greater than for the other states.

Yet another finding of interest can be seen by a comparison of the responses for States 3 and 6. The lone feature seen in State 3 for deoxyHb (also totalHb) images corresponds closely to the radial vein. Inspection of State 6 images (hyperemia) reveals a large amplitude response in close proximity of the radial artery (7 o'clock position) that has an opposite algebraic sign. Integration of these image features, for example, as would occur by an equivalent fMRI measurement or by DOT, as it is usually practiced, would produce a cancellation of features, leading to loss of information. In fact, because many of the individual components of the indicated autoregulatory states have opposite signs, it is evident that many other features could be similarly unrecognized by these techniques depending on the nature of the manipulation introduced.

These considerations are documented explicitly by results shown in FIG. 4. Here the image maps corresponding to findings by DOT and the fMRI equivalent result (i.e., deoxyHb) are shown for the data presented in FIG. 2. Comparison of the image features across the hemoglobin states (i.e., oxyHb, deoxyHb, totalHb) reveals some differences (notably amplitude), but are much less revealing than the image features revealed in FIG. 3.

Returning to FIG. 3, still other valuable information can be gleaned from examination of the FIA image maps. For instance, comparison of the time fraction maps (panels 19-24) to the Hb feature maps (Panels 1-18) reveals that while some areas may experience greater durations in a given state, the magnitude of the response can be significantly different from other areas. This is revealed well in the maps for State 6. Here we see that while the interior of the arm has the largest duration in State 6, the largest magnitude of Hb response is in the periphery of the arm. This finding is suggestive of tissue-dependent differences in metabolic activity that serves to elicit a differential reactive hyperemia response.

It can be useful to reduce the information content revealed by the image maps in FIG. 3 to single time-series tracing for each of the six autoregulatory states. Results in FIG. 5A show an example of this calculation, wherein the integration performed produces a time-series of the time-dependence of the volume fraction of the image map for each autoregulatory state. Inspection shows that prior to inflation of the cuff, >90% of the image volume is confined to States 1 (blue line) and 4 (yellow line), (i.e., oxygen balance). This finding is reasonable given that the tissue is initially at rest. Inflation of the cuff, produces an abrupt decrease in State 1 with an rapid increase in State 4 to approximately 90% of the total image volume indicating the venous congestion is occurring. Because this maneuver does not block inflow of arterial blood, hemoglobin desaturation generally does not occur (low values for States 2 (red), and 3 (green)). Upon release of the cuff, State 4 values rapidly decline. Unlike the onset period, the recovery period, however, displays much more complex behavior. For instance, first to respond is a transient rise in State 3. Also seen are transient responses in other states (State 2, 5) but with different time delays. Notable is a large amplitude increase in State 6 indicating that some degree of reactive hyperemia has occurred. Also seen is that accompanying the decline in this state is a rise in State 1 suggestive of tissue recovery.

For comparative purposes, a similar data analysis has been performed on the data collected from the cuff ischemia experiment. These results are shown in FIG. 5B.

Inspection reveals a response profile that, with few exceptions, is entirely different from that seen in response to venous congestion. One of these exceptions is the magnitude of the initial responses seen for States 1 and 4. Here we see that prior to cuff inflation, the tissue is mainly in oxygen balance, which is the expected response at rest. Also similar is the rise in State 1 during the recovery period. In contrast to the results shown in FIG. 5A, here we see that upon inflation, there is an abrupt rise in State 2 (uncompensated oxygen debt) associated with a decline in State 1. All other states remain low during the ischemic period. Upon release of the cuff the volume of tissue in State 2 declines precipitously, but slower than the decline seen for State 4 during recovery from venous congestion (FIG. 4). This is reasonable, as the latter involves washout of the major veins, while the former requires washout of the microvascular bed. Also seen during the recovery period are transient elevations in the other states, notably States 3-6, but with different amplitudes and slower time courses compared to that seen during recovery from venous congestion.

Whereas the information revealed in FIGS. 2-5 were based on time or spatial integration of image findings, it is apparent to those skilled in the art that useful information can also be identified by inspection of image finding made at discrete points in time. An example of this finding is shown in FIG. 6 from measurements made from the head of a freely moving rat. Shown is a volume rendered image revealing locations in a 3D volume that exist in the different autoregulatory state indicated in FIG. 1. The indication of discrete, mainly nonverlapping, volumes is suggestive of the occurrence of a cyclical process associated brain perfusion, a finding consistent with fMRI studies.

Conclusion: Experimental results here demonstrate that this invention is able to provide six sub-stage of deoxyhemoglobin, oxyhemoglobin, and total hemoglobin (spatial and temporal) comparing to current available technologies and methods such as fMRI and NIRS. Such detailed and enhanced information is very valuable in various applications.

These results, while emphasizing responses associated with the process of vascular autoregulation, are representative of similar findings that can be obtained on other kinds of tissue metabolism.

This invention is directed to method comprising the steps of: applying at least one sensor to a portion of a subject's body; directing at least one energy source at a portion of the subject's body; detecting the emitted energy signal from at least one sensor; processing said data; and producing at least one image or at least a time series or a combination thereof, to delineate variations of tissue metabolism, wherein the image can be a topographic, 2D tomographic, 3D tomographic map or any of the combination thereof.

This invention also directs to a process for deriving information on tissue metabolism in a subject's body to produce at least one image or a time serious comprising the steps of: 1) collecting data from at least one sensor; 2) normalizing collected data to an experimental or computed mean value; 3) producing at least one parameter map having at least one pixel value from the normalized data using indirect imaging methods, or computing at least one image map from the normalized data and converting to at least one parameter map having at least one pixel value; 4) comparing pixel values of the parameter map in step (3) to their respective mean value and categorizing such values according to whether the parameter value is above or below its mean value; and 5) computing a parameter map of categorized pixel data and optionally producing a time series comprising a step of computing a spatial mean value for each parameter at each time point; wherein the image can be a topographic, 2D tomographic, 3D tomographic map or any combination thereof.

This invention further directs to a process for deriving information on tissue metabolism in a subject's body to produce at least one image, at least a time series, a combination thereof comprising the steps of: (1) collecting data from at least one sensor; (2) producing at least one parameter map having at least one pixel value from the collected data using indirect imaging methods, or generating at least one image map from the collected data using said indirect imaging methods and converting to at least one parameter map having at least one pixel value; (3) normalizing pixel values in Step (2) to an experimental or computed mean value either after generation of the parameter map(s) or prior to conversion to said parameter map; (4) comparing pixel values of at least one parameter map in Step (3) to their respective mean value and categorizing such values according to whether the parameter value is above or below its mean value; and (5) computing a parameter map of categorized pixel value and optionally producing a time series comprising a step of computing a spatial mean value for each parameter at each time point, wherein the image can be a topographic, 2D tomographic, 3D tomographic map, or any combination thereof.

This invention also directs to a process for deriving information on tissue metabolism in a subject's body to produce at least one time series comprising the steps of: (1) collecting data from at least one sensor, (2) normalizing collected data to an experimental or computed mean value followed by computation of a parameter value or computing the parameter value from collected data followed by normalization of the resultant time series; (3) Comparing the values in Step (2) to their respective mean value and categorizing such values according to whether the parameter value is above or below its mean value.

Preferably, the tissue metabolism is associated with hemoglobin.

Preferably, the tissue metabolism further comprises a contrast agent.

This invention also directs to a method of further comparing said detailed information to clinic, healthcare, research facilities, and pharmaceutical industry for various applications purposes.

1. A method for producing at least one image to delineate variations of tissue metabolism comprising the steps of:
applying at least one sensor to a portion of a subject's body;
directing at least one energy source at a portion of the subject's body;
detecting the emitted energy signal from said sensor;
processing said data, and producing at least one image or derivative information;
wherein the image can be a topographic, 2D tomographic, 3D tomographic map or any of the combination thereof.

2. A method for delineating at least one time-variation in tissue metabolism comprising the steps of:
applying at least one sensor to a portion of a subject's body;
directing at least one energy source at a portion of the subject's body;
detecting the emitted energy signal from said sensor;
processing said data, and producing at least one time series.

3. A method for producing at least one image map or at least one time-series to delineate variations in tissue metabolism associated with hemoglobin, comprising the step of:
applying at least one sensor to a portion of a subject's body;
directing at least one energy source at a portion of the subject's body;
detecting the emitted energy signal from said sensor;
processing said data, and producing at least one image and/or at least one processed time series.
wherein the image can be a topographic, 2D tomographic, 3D tomographic map or any of the combination thereof.

4. The method of claims 1 and 2, wherein a combination of at least one image and at least one time series is produced.

5. The method of claims 1 to 3, wherein the energy source comprises at least one wavelength.

6. The method of claims 1 to 3, wherein the energy source can be monochromatic or polychromatic or any combination thereof.

7. The method of claims 1 to 3, wherein the energy source can be operated in a continuous wave, or a frequency domain, or a time domain, or any combination thereof.

8. The method of claims 1 to 3, wherein the energy source can be inside or outside or on the subject's body.

9. The method of claims 1 to 3 and 11-13, wherein the sensor measures a photo-acoustic signal, or modulation of light produced by focused ultrasound, or a fluorescent signal, or any combination thereof.

10. The method of claims 1-3 and 11-13, wherein the detection of the tissue metabolism further comprises a contrast agent.

11. A process for deriving information on tissue metabolism in a subject's body to produce at least one image and/or derivative information comprising the steps of:
1) collecting data from at least one sensor;
2) normalizing collected data to an experimental or computed mean value;
3) producing at least one parameter map having at least one pixel value from the normalized data using indirect imaging methods, or computing at least one image map from the normalized data and converting to at least one parameter map having at least one pixel value;
4) comparing pixel values of the parameter map in step (3) to their respective mean value and categorizing such values according to whether the parameter value is above or below its mean value; and
5) computing a parameter map of categorized pixel value and optionally producing at least one time series comprising a step of computing a spatial mean value for each parameter at each time point;
wherein the image can be a topographic, 2D tomographic, 3D tomographic map or any combination thereof.

12. A process for deriving information on tissue metabolism in a subject's body to produce an image and/or derivative information comprising the steps of:
(1) collecting data from at least one sensor;
(2) producing at least one parameter map having at least one pixel value from the collected data using indirect imaging methods, or generating at least one image map from the collected data using said indirect imaging methods and converting to at least one parameter map having at least one pixel value;
(3) normalizing pixel values in Step (2) to an experimental or computed mean value either after generation of at least one parameter map(s) or prior to conversion to said parameter map;
(4) comparing pixel values of at least one parameter map in Step (3) to their respective mean value and categorizing such values according to whether the parameter value is above or below its mean value; and
(5) computing at least one parameter map of categorized pixel data and optionally producing at least one time series comprising a step of computing a spatial mean value for each parameter at each time point,
wherein the image can be a topographic, 2D tomographic, 3D tomographic map, or any combination thereof.

13. A process for deriving information on tissue metabolism in a subject's body to produce at least one time series comprising the steps of:
(1) collecting data from at least one sensor,
(2) normalizing collected data to an experimental or computed mean value followed by computation of a parameter value or computing the parameter value from collected data followed by normalization of the resultant time series;
(3) comparing the values in Step (2) to their respective mean value and categorizing such values according to whether the parameter value is above or below its mean value.

14. The method of claims 1-3 and 11-12, wherein the image is formed by a temporal, non-temporal mean or any combination thereof.

15. The method of claims 1-3 and 11-13, wherein the collected data is a measure of a fraction of the incident energy or is a measure of incident energy that has been converted to another energy form, or combined.

16. The method of claims 1-3 and 11-13, wherein the sensor can be silicon photodiodes, avalanche, photo-multipliers (PMT), CCD, CID, streak camera, an acoustic sensor or any combination thereof.

17. The method of claims 1-3 and 11-13, wherein the sensor can be directly placed on the subject's body or inside the subject's body, remotely, or any combination thereof.

18. The process of claims 12-14, wherein the tissue metabolism is associated with hemoglobin.

19. The method of claims 1-3, 12-14, wherein the method further comprises a step of comparing said image or time series for
